(19) United States
(12) Patent Application Publication
(10) Pub. No.: US 2025/0377530 A1
(43) Pub. Date: Dec. 11, 2025

(54) OPTICAL OBSERVATION SYSTEM HAVING A PIECE OF OPTICAL OBSERVATION EQUIPMENT AND AN ILLUMINATION DEVICE, AND METHOD FOR IDENTIFYING ON AN OBJECT REGION AN IMAGED PORTION OF SAID OBJECT REGION

(71) Applicant: Carl Zeiss Meditec AG, Jena (DE)

(72) Inventors: Andre MUELLER, Koenigsbronn-Zang (DE); Artur HOEGELE, Oberkochen (DE)

(21) Appl. No.: 19/231,705

(22) Filed: Jun. 9, 2025

(30) Foreign Application Priority Data
Jun. 11, 2024 (DE) 10 2024 116 325.9

(51) Int. Cl. G02B 21/06; A61B 90/00; A61B 90/35; G02B 21/00

(52) U.S. Cl. CPC G02B 21/06; A61B 90/35; A61B 90/361; G02B 21/0012; A61B 2090/363; A61B 2090/373

(57) ABSTRACT

An illumination device for a piece of optical observation equipment is provided. The illumination device is equipped with at least one illumination beam path that leads from an illumination light source to an observation object and serves to generate an illuminated field on the observation object by illumination light, and a projection device for projecting a structure onto the observation object. The projection device acts on the illumination light in such a way that the structure is projected onto the observation object within the illuminated field with the aid of the illumination light that generates the illuminated field. A method is provided for identifying on an observation object an object region of said observation object that has been imaged on an image sensor of a piece of optical observation equipment. In the method, a structure is projected onto the observation object.

The present invention relates to an optical observation system having a piece of optical observation equipment and an illumination device for a piece of optical observation equipment. In addition, the invention also relates to the piece of optical observation equipment of the optical observation system and to the illumination device of the optical observation system. The invention furthermore relates to a method for identifying on an object region a portion of said object region imaged on an image sensor of a piece of optical observation equipment.

Targeted alignment of a piece of optical observation equipment with an observation object to be imaged can be difficult. For example, surgical staff may find it difficult to align a surgical microscope before or during a surgical procedure. This is particularly true when a digital surgical microscope is used, in which the surgical staff does not look through eyepieces but at a monitor on which an imaged object region of the observation object is displayed. Since the monitor does not move when the surgical microscope is moved, the orientation of the monitor is completely independent of the orientation of the surgical microscope. This means that surgical staff lose the direct relationship between the imaged object region and the position and orientation of the latter in the observation object—a relationship that they would have had if they were viewing the observation object using eyepieces on the surgical microscope. In addition, the size of the object region shown on the monitor depends on the magnification set on the surgical microscope and the working distance between surgical microscope and observation object. All of this makes it difficult for the treating surgical staff to identify the orientation and size of the imaged object region in the observation object shown on the monitor.

Although the position of the imaged object region can be identified on the basis of the position of the illuminated field used to illuminate the observation object, the generally round illuminated field does not allow identification on the observation object of the orientation of the object region that is imaged on the image sensor. If the size of the illuminated field does not change with a change in the magnification of the image representation, the illuminated field also does not allow estimation of the size of the imaged object region.

U.S. Pat. No. 11,439,477 B2 therefore proposes to provide a surgical microscope not only with its illumination unit but also with a light projection unit that projects a light pattern, such as a frame, onto the observation object outside of and at a distance from the imaged object region, and this provides an indication of the orientation and the size of the imaged object region in the observation object. In an alternative embodiment variant, U.S. Pat. No. 11,439,477 B2 proposes the omission of the additional projection unit and, instead, the generation of an additional luminous point, which indicates the orientation of the imaged object region in the observation object, outside of the actual illuminated field by means of the illumination unit.

In comparison with this prior art, a first problem addressed by the present invention is that of providing an advantageous illumination device for a piece of optical observation equipment and also a piece of optical observation equipment and an optical observation system having an advantageous illumination device.

A second problem addressed by the present invention is that of providing an advantageous method for identifying on an observation object an object region of said observation object that has been imaged on an image sensor of a piece of optical observation equipment.

The first problem is solved by an illumination device as claimed in claim 1, by a piece of optical observation equipment as claimed in claim 6 and by an optical observation system as claimed in claim 10. The second problem is solved by a method as claimed in claim 11. The dependent claims contain advantageous configurations of the invention.

An illumination device according to the invention for a piece of optical observation equipment comprises at least one illumination beam path that leads from an illumination light source to an observation object and serves to generate an illuminated field on the observation object by means of illumination light. Two beam paths may also be present in the case of coaxial illumination. Coaxial illumination is used in stereoscopic optical observation equipment and means that the illumination of the observation object is coaxial with respect to the stereoscopic observation beam paths. The illumination beam paths of the coaxial illumination thus each represent a partial beam path of the coaxial illumination. The at least one illumination light source may be a primary light source such as e.g. an incandescent or gas discharge lamp, the output of a light guide, a self-luminous display such as for instance an LCD, LED or OLED display, etc. Alternatively, the illumination light source may be a secondary light source, i.e. the image of a primary light source.

In addition, the illumination device according to the invention comprises a projection device for projecting a structure onto the observation object. This projection device acts on the illumination light in such a way that the structure is projected onto the observation object within the illuminated field with the aid of the illumination light that generates the illuminated field.

An additional light projection unit as proposed in U.S. Pat. No. 11,439,477 B2 is not required as a result of the projection device acting on the illumination light in order to project the structure onto the observation object. In addition, unlike the second alternative described in U.S. Pat. No. 11,439,477 B2, no further luminous spot is generated outside the actual illuminated field. Such an additional luminous spot might potentially cause bothersome stray light.

The illumination device according to the invention allows the implementation of the method according to the invention for identifying on an observation object an object region of said observation object that has been imaged on an image sensor of a piece of optical observation equipment, in which a structure that represents an indicator for the object region of the observation object imaged on the image sensor is projected onto the observation object with the aid of illumination light that generates an illuminated field on the observation object. The identification on the observation object of the object region that is imaged on the image sensor makes it easier for an operator to set a specific orientation of the imaged object region in the observation object or to identify the imaged object region in the observation object.

The projection device may be configured to act on the illumination light in such a way that there is a structure-creating shadowing in the illuminated field. For example, the shadowing may be brought about by a stop arranged in or near to a plane of the illumination beam path conjugate to the plane of the illuminated field. When the stop is arranged in a plane of the illumination beam path conjugate to the plane of the illuminated field, the illumination beam path images the structure of the stop into the illuminated field plane, i.e. the illumination beam path creates an image of the stop in the illuminated field plane. The farther the plane in which the stop is located is from the plane of the observation beam path conjugate to the plane of the illuminated field, the more blurred the image of the stop becomes, until eventually it becomes unrecognizable. Thus, the plane in which the stop is arranged should be considered to be near to a plane conjugate to the plane of the illuminated field if the image of the stop in the plane of the illuminated field is blurred but still recognizable. Since the image of the stop is only used to indicate the orientation and optionally the size of the object region that is imaged on the sensor, and this is sufficiently possible even with a blurred image of the stop, blurring in the image of the stop is generally not considered to be bothersome. The shadowing can be created by simple means by arranging a shadowing-inducing stop in or near to a plane of the illumination beam path conjugate to the plane of the illuminated field. In particular, a stop changer device such as e.g. a stop wheel or a stop slider having a plurality of stops that differ from one another in terms of their shape and/or their size may be present in or near to the plane of the illumination beam path conjugate to the plane of the illuminated field. The use of a plurality stops arranged in a stop changer device for example allows the use of simple means to project structures of different sizes onto the observation object, and so the projected structure can also be an indicator for the magnification of the piece of optical observation equipment. Should it be possible to change the magnification in increments in a piece of optical observation equipment to which the illumination device is assigned, the sizes of the stops may be matched to the increments of the magnification of the piece of optical observation equipment. Should the piece of optical observation equipment allow a continuous change in magnification, the stop coming closest to the size of the imaged object region can be selected. Fundamentally, however, there is also the option of integrating into the illumination beam path a zoom system, by means of which the image representation of the stop in the illuminated field can be magnified continuously. The filter wheel may be omitted in that case, or the filter wheel can be used to provide different stop geometries. However, instead of the stop wheel or the stop slider, it is easier to provide a stop changer device in the form of a transilluminated display on which a stop structure is displayed. Any desired structure can be displayed on the transilluminated display, and so the display can be used to easily realize stops that differ from one another in terms of shape and/or size. If the display is a color display, it is also possible to provide color-coded information, for example by virtue coloring one side in a specific color in order to be able to clearly specify a specific orientation. However, a grayscale display is completely sufficient for projecting the structure.

In a further alternative configuration, the illumination device may contain a self-luminous display as the at least one illumination light source. This self-luminous display is then arranged in or near to a plane of the illumination beam path conjugate to the plane of the illuminated field. The structure can then be displayed on the display, like in the case of the transilluminated display. In this context, the self-luminous display may be a grayscale display or a color display. Since the plane of the display is conjugate to the plane of the illuminated field or located near to a plane conjugate to the plane of the illuminated field, the illumination beam path images an image displayed on the display into the illuminated field plane. A structure displayed on the display is therefore to be identified as image of the structure in the illuminated field, wherein the image of the structure may also be blurred if the plane of the display is not located in but only near to the plane conjugate to the plane of the illuminated field. As a rule, this is unproblematic as long as the structure can be identified despite the blur. Since a display can display a multiplicity of structures in a multiplicity of sizes, this alternative offers a very high degree of flexibility when projecting the structures and matching the projected structures to the orientation and/or size of an imaged object portion. In order to obtain the most homogeneous illuminated field possible, the self-luminous display should have the most homogeneous luminance distribution possible.

A piece of optical observation equipment according to the invention comprises an illumination device according to the invention and at least one imaging beam path that generates an image of an object region of the observation object. The at least one imaging beam path may lead to an image sensor for recording images of the object region. In the case of multiple imaging beam paths, i.e. at least two imaging beam paths, at least one may lead to an image sensor. At least one further one may then lead to a further image sensor or to an eyepiece. Such a piece of optical observation equipment allows an operator to more easily identify the orientation of the piece of optical observation equipment in relation to the observation object by virtue of the fact that the orientation and optionally the size of an imaged object region in the observation object can be projected onto the observation object with the aid of the illumination device according to the invention. Should images be recorded by means of an image sensor, the structure projected onto the observation object may represent an indicator for the object region that is imaged on the image sensor. In particular, it may represent the shape, the size, and the orientation of the object region that is imaged on the image sensor. For example, the orientation of the object region that is imaged on the image sensor may be specified by virtue of the structure projected onto the observation object containing a marking, by means of which it is possible to identify the orientation of the image sensor in relation to the observation object.

According to a further aspect of the invention, provision is made for an optical observation system having a piece of optical observation equipment according to the invention and a positioning apparatus for positioning the piece of optical observation equipment in relation to the observation object. The positioning apparatus comprises at least one brake which can be put into an activated and a deactivated state, wherein the brake in the activated state blocks a movement of the piece of optical observation equipment in relation to the observation object and does not block said movement in the deactivated state. Moreover, the optical observation system comprises a controller connected to the positioning apparatus in order to receive a status signal representing the state of at least one brake and connected to the illumination device in order to output an activation signal for activating the projection of the structure onto the observation object. The connection between controller and illumination device may be either indirect, by virtue of acting on a microscope controller that controls all the optical observation equipment including the illumination device in order to cause this to activate the projection of the structure onto the observation object, or direct, by virtue of itself acting directly on at least one component of the illumination device in order to activate the projection of the structure onto the observation object. The controller is configured to output the activation signal only if the status signal represents a deactivated state of the brake.

A deactivated brake generally means that the position and/or orientation of the piece of optical observation equipment should be changed. When using the optical observation system according to the invention, the method according to the invention for identifying on an observation object an object region of said observation object that has been imaged on an image sensor of a piece of optical observation equipment may be further developed in such a way that the structure is projected onto the observation object when at least one brake serving for a change in the position and/or the orientation of the piece of optical observation equipment in relation to the observation object is deactivated. In that case, the projecting of the structure onto the observation object may be terminated again at the latest when all brakes are activated. As a rule, activating all brakes means that the change in the position and/or the orientation of the piece of optical observation equipment in relation to the observation object has been terminated, and the actual work with the piece of optical observation equipment begins.

Should the positioning apparatus be designed for manually moving the piece of optical observation equipment in relation to the observation object, a motion detector for detecting a movement of the piece of optical observation equipment may be present in addition or in an alternative. In that case, the controller is connected to the motion detector in order to receive a movement status signal representing the movement state of the piece of optical observation equipment and connected to the illumination device in order to output an activation signal for activating the projection of the structure onto the observation object. In addition, it is then configured to output the activation signal only if the movement status signal represents a movement of the piece of optical observation equipment. Especially in the case of positioning apparatuses without brakes, this can be used to bring about automatic projection during the movement of the piece of optical observation equipment.

However, of course, there naturally is also the possibility that the projecting of the structure is started and/or terminated by way of an operator input, for example by way of a touch screen, by way of a voice command or by way of actuating a switch on the piece of optical observation equipment or a switch, for instance a foot switch, connected to the piece of optical observation equipment. Starting and/or terminating the projection by way of an operator input can in particular also be implemented as a supplement to an automatic start and/or termination of the projection, wherein the operator input may in that case have a higher priority than the automatic start and/or the automatic termination of the projection. In this way, an operator can for example terminate a projection that was automatically started on account of the deactivation of a brake or a detected movement of the piece of optical observation equipment because said projection is not required or because said projection is a distraction. Likewise, the projection may start following an operator input in order to visualize the orientation and/or the size of an object region, for instance represented on a monitor, although there has been no change in the position and/or the orientation.

Further features, properties and advantages of the present invention are explained below on the basis of an exemplary embodiment and with reference to the appended figures.

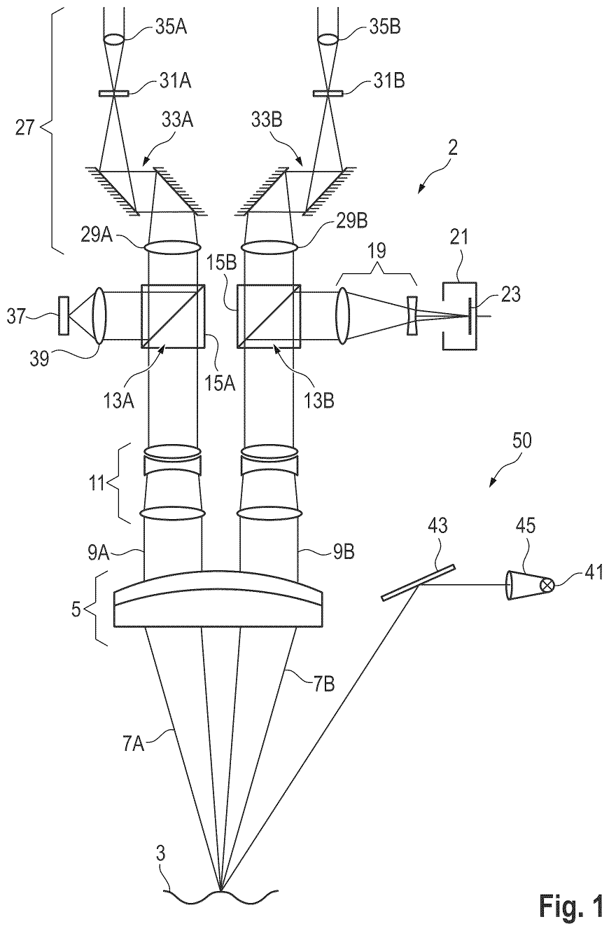

FIG. 1 shows a schematic illustration of a surgical microscope.

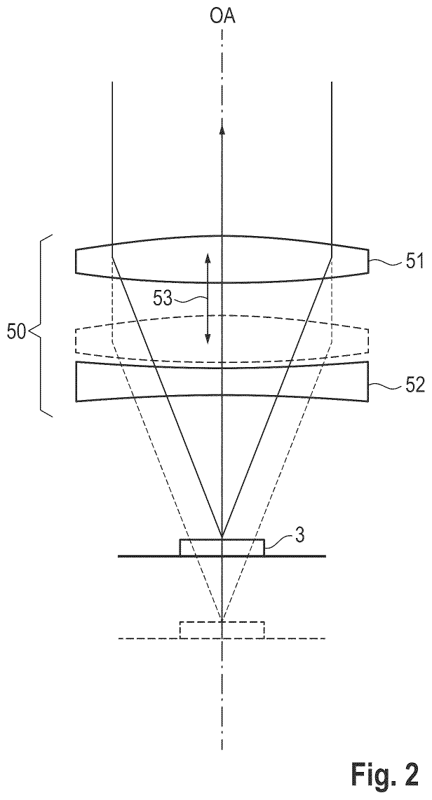

FIG. 2 shows an objective that can be used to vary the working distance of a surgical microscope.

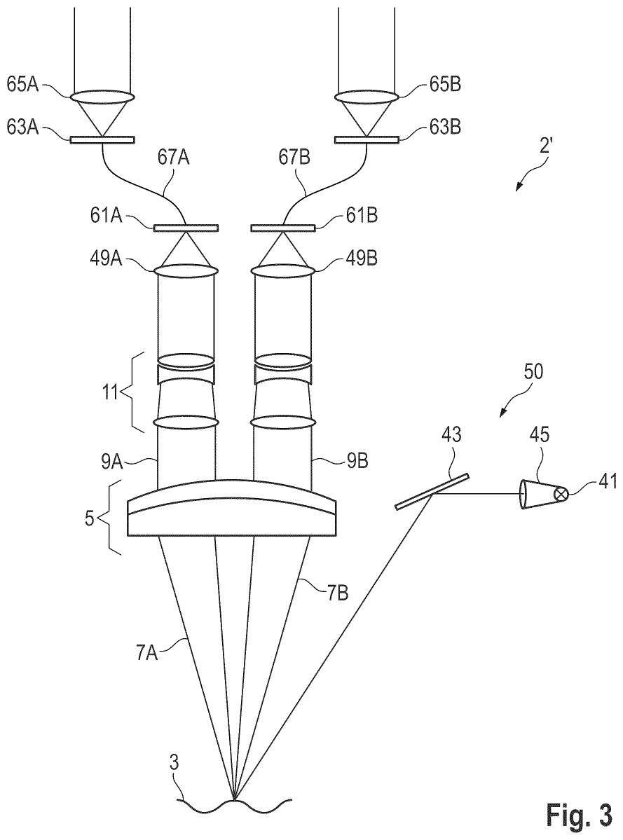

FIG. 3 shows a schematic illustration of a purely digital surgical microscope.

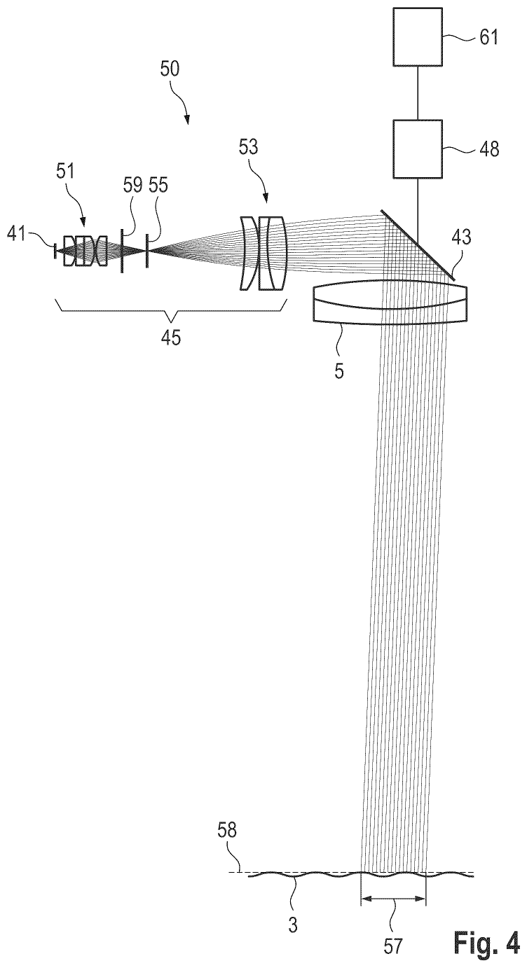

FIG. 4 shows an illumination device and an associated illumination beam path.

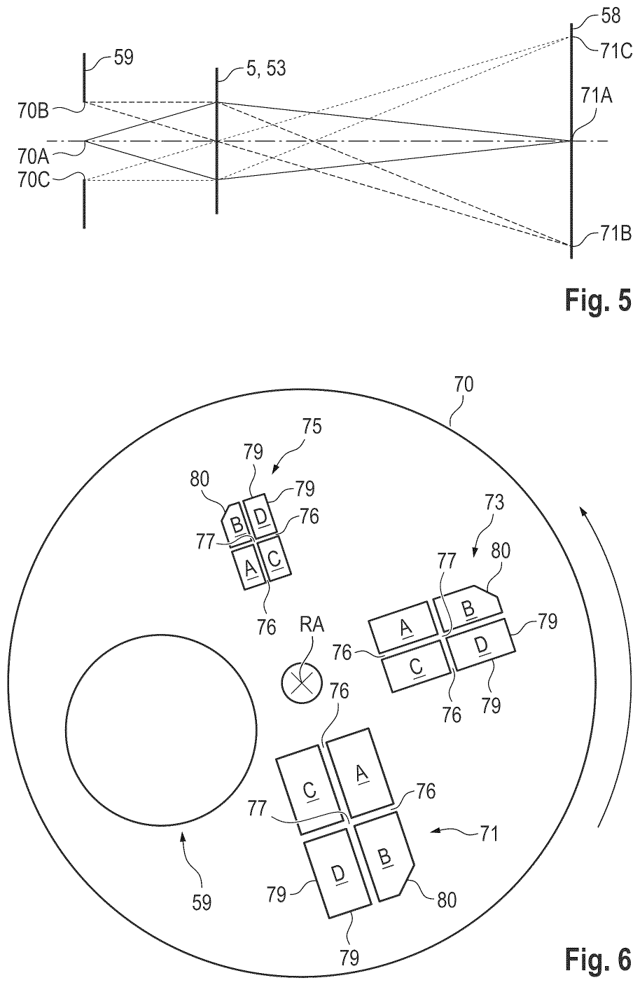

FIG. 5 shows the image representation of a field stop in the plane of an illuminated field.

FIG. 6 shows a stop wheel with multiple stops.

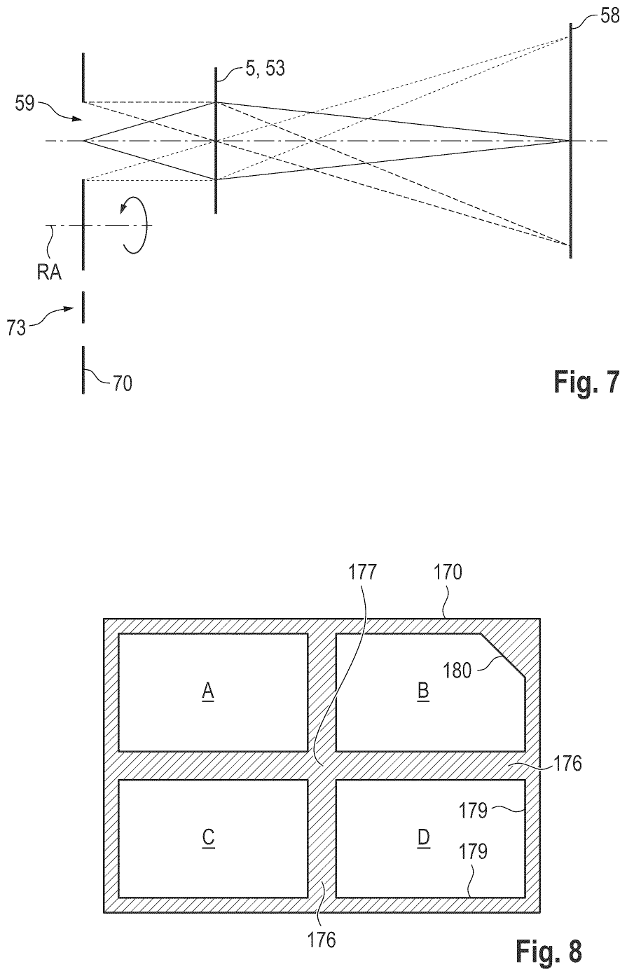

FIG. 7 shows the drawing from FIG. 5, but with a stop wheel instead of the field stop.

FIG. 8 shows a display with a structure shown thereon.

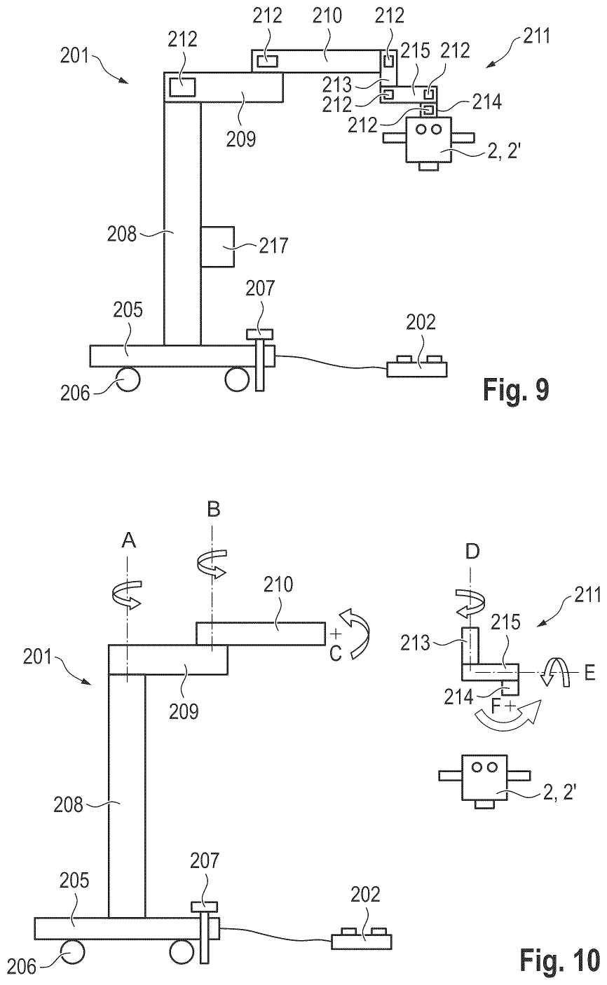

FIG. 9 shows a surgical microscope attached to a stand.

FIG. 10 shows the movement options for the surgical microscope from FIG. 9.

A surgical microscope 2 as an exemplary embodiment of a piece of optical observation equipment is described below with reference to FIG. 1. As essential component parts, the surgical microscope 2 shown in FIG. 1 comprises an objective 5 that faces an observation object 3 and may be embodied as an achromatic or apochromatic objective in particular. In the present exemplary embodiment, the objective 5 consists of two partial lenses that are cemented to one another and together form an achromatic objective 5.

The observation object 3 is arranged in the focal plane of the objective 5 such that it is imaged at infinity by the objective 5. Expressed differently, a divergent beam 7A, 7B emanating from the observation object 3 is converted into a parallel beam 9A, 9B during its passage through the objective 5. The illustrated surgical microscope 2 is a piece of stereoscopic optical observation equipment. The beams 7A, 7B and 9A, 9B therefore define stereoscopic partial beam paths of the surgical microscope 2.

A magnification changer 11 is arranged on the observer side of the objective 5 and may be embodied either as a zoom system for changing the magnification factor in a continuously variable manner or as what is known as a Galilean changer for changing the magnification factor in a stepwise manner. In a zoom system, constructed by way of example from a lens combination having three lenses, the two object-side lenses may be displaced in order to vary the magnification factor. In actual fact, however, the zoom system also may comprise more than three lenses, for example four or more lenses, in which case the outer lenses then may also be arranged in a fixed manner. In a Galilean changer, by contrast, there are a plurality of fixed lens combinations which represent different magnification factors and which can be introduced into the stereoscopic partial beam paths defined by the component beams 9A, 9B in alternation. Both a zoom system and a Galilean changer convert an object-side parallel beam into an observer-side parallel beam with a different beam diameter. In the present exemplary embodiment, the magnification changer 11 is already part of the binocular beam path of the surgical microscope 2, i.e. it has a dedicated lens combination for each stereoscopic partial beam path 9A, 9B of the surgical microscope 2. In the present exemplary embodiment, a magnification factor is set by means of the magnification changer 11 by way of a motor-driven actuator which, together with the magnification changer 11, is part of a magnification changing unit for setting the magnification factor.

The magnification changer 11 is adjoined on the observer side by an interface arrangement 13A, 13B, by means of which external equipment may be connected to the surgical microscope 2 and which comprises beam splitter prisms 15A, 15B in the present exemplary embodiment. However, other types of beam splitters may also be used in principle, for example partly transmissive mirrors. In the present exemplary embodiment, the interfaces 13A, 13B serve to output couple a beam from the stereoscopic partial beam path 9B of the surgical microscope 2 (beam splitter prism 15B) and to input couple a beam into the stereoscopic partial beam path 9A of the surgical microscope 2 (beam splitter prism 15A).

In the present exemplary embodiment, the beam splitter prism 15A in the stereoscopic partial beam path 9A serves to reflect information or data for an observer via the beam splitter prism 15A into the stereoscopic partial beam path 9A of the surgical microscope 2 with the aid of a display 37, for example a digital mirror device (DMD) or an LCD display, and an associated optical unit 39. A camera adapter 19 with a camera 21 fastened thereto, said camera being equipped with an electronic image sensor 23, for example with a CCD sensor or a CMOS sensor, is arranged at the interface 13B in the other stereoscopic partial beam path 9B. By means of the camera 21, it is possible to record an electronic image, and in particular a digital image, of the tissue region 3, for instance for documentation purposes or for displaying an image of the observation object 3 on a monitor. However, when viewing the images on a monitor, surgical staff lose the direct relationship between the imaged object region and the position and orientation of the latter in the observation object 3—a relationship that they would have had if they were viewing the observation object through the binocular tube.

A binocular tube 27 adjoins the interface 13 on the observer side. It has two tube objectives 29A, 29B, which focus the respective parallel beam 9A, 9B on an intermediate image plane 31, i.e. image the observation object 3 onto the respective intermediate image plane 31A, 31B. Finally, the intermediate images situated in the intermediate image planes 31A, 31B are imaged in turn at infinity by eyepiece lenses 35A, 35B, and so a viewer can view the intermediate image with a relaxed eye. Moreover, an increase in the distance between the two component beams 9A, 9B is implemented in the binocular tube by means of a mirror system or by means of prisms 33A, 33B in order to adapt said distance to the interocular distance of the viewer.

The surgical microscope 2 moreover is equipped with an illumination device 50, by means of which the observation object 3 may be illuminated with broadband illumination light. To this end, the illumination device 50 in the present exemplary embodiment comprises a white-light source 41, for example a halogen lamp or a gas discharge lamp. The light emanating from the white-light source 41 is steered in the direction of the observation object 3 via a deflection mirror 43 or a deflection prism in order to illuminate said observation object. Furthermore, an illumination optics unit 45 is present in the illumination device 50 and ensures uniform illumination of the entire observed observation object 3. The illumination device 50 will be explained in more detail below with reference to FIGS. 4 and 5.

Reference is made to the fact that the illumination beam path depicted in FIG. 1 is highly schematic and does not necessarily reproduce the actual course of the illumination beam path. In principle, the illumination beam path may take the form of what is known as oblique illumination, which comes closest to the schematic illustration in FIG. 1. In such oblique illumination, the beam path extends at a relatively large angle (6° or more) with respect to the optical axis of the objective 5 and, as illustrated in FIG. 1, may extend completely outside the objective. The illumination angle changes inter alia with the working distance and may also be less than 6° if the working distance is large. In an alternative, however, there is also the option of allowing the illumination beam path of the oblique illumination to extend through a marginal region of the objective 5. A further option for the arrangement of the illumination beam path is what is known as 0° illumination, in which the illumination beam path extends through the objective 5 and is input coupled into the objective between the two partial beam paths 9A, 9B, along the optical axis of the objective 5 in the direction of the observation object 3. Finally, there is also the option of embodying the illumination beam path as what is known as coaxial illumination, in which a first illumination partial beam path and a second illumination partial beam path are present. The partial beam paths are input coupled into the surgical microscope in a manner parallel to the optical axes of the observation partial beam paths 9A, 9B by way of one or more beam splitters such that the illumination extends coaxially in relation to the two observation partial beam paths.

In the embodiment variant of the surgical microscope 2 shown in FIG. 1, the objective 5 consists only of one achromatic lens. However, use can also be made of an objective lens system composed of a plurality of lenses, in particular what is known as a zoom lens, by means of which it is possible to vary the working distance of the surgical microscope 2, i.e., the distance between the object-side focal plane and the vertex of the first object-side lens surface of the objective 5, also referred to as front focal distance. The observation object 3 arranged in the focal plane is imaged at infinity by the zoom lens 50, too, and so a parallel beam is present on the observer side.

One example of a zoom lens is depicted schematically in FIG. 2. The zoom lens 50 comprises a positive member 51, i.e. an optical element with positive refractive power, depicted schematically as a convex lens in FIG. 2. Moreover, the zoom lens 50 comprises a negative member 52, i.e. an optical element with negative refractive power, depicted schematically as a concave lens in FIG. 2. The negative member 52 is situated between the positive member 51 and the observation object 3. In the depicted zoom lens 50, the negative member 52 has a fixed arrangement, whereas, as indicated by the double-headed arrow 53, the positive member 51 is arranged to be displaceable along the optical axis OA. When the positive member 51 is displaced into the position illustrated by dashed lines in FIG. 2, the back focal length increases, and so there is a change in the working distance of the surgical microscope 2 from the observation object 3.

Even though the positive member 51 has a displaceable configuration in FIG. 2, it is also possible, in principle, to arrange the negative member 52 to be movable along the optical axis OA instead of the positive member 51. However, the negative member 52 often forms the last lens element of the zoom lens 50. A stationary negative member 52 therefore offers the advantage of making it easier to seal the interior of the surgical microscope 2 from external influences. Furthermore, it is noted that, even though the positive member 51 and the negative member 52 in FIG. 2 are only illustrated as individual lens elements, each of these members may also be realized in the form of a lens group or a cemented element instead of in the form of an individual lens element, for example in order to design the zoom lens to be achromatic or apochromatic.

A digital surgical microscope 2′ as a further exemplary embodiment of a piece of optical observation equipment is described below with reference to FIG. 3. In the digital surgical microscope 2′, the main objective 5, the magnification changer 11, which merely represents an option in the digital surgical microscope 2′ and hence need not necessarily be present, and the illumination system 41, 43, 45 do not differ from the surgical microscope 2 with an optical viewing unit, depicted in FIG. 1. The difference lies in the fact that the surgical microscope 2′ shown in FIG. 3 does not comprise an optical binocular tube. Instead of the tube objectives 29A, 29B from FIG. 1, the surgical microscope 2′ from FIG. 3 comprises focusing lenses 49A, 49B with which the binocular observation beam paths 9A, 9B are imaged onto digital image sensors 61A, 61B. Here, the digital image sensors 61A, 61B can be e.g. CCD sensors or CMOS sensors. The images recorded by the image sensors 61A, 61B are transmitted to digital displays 63A, 63B, which may be embodied as LED displays, as LCD displays, or as displays based on organic light-emitting diodes (OLEDs). As in the present example, eyepiece lenses 65A, 65B can be assigned to the displays 63A, 63B, by means of which lenses the images presented on the displays 63A, 63B are imaged at infinity such that a viewer can view said images with relaxed eyes. The displays 63A, 63B and the eyepiece lenses 65A, 65B may be part of a digital binocular tube; however, they may also be part of a head-mounted display (HMD) such as for instance a pair of smartglasses. Even though FIG. 3 shows a transmission of the images recorded by the image sensors 61A, 61B to the displays 63A, 63B of a digital binocular tube by means of cables 67A, 67B, the images may also be transmitted wirelessly to the displays 63A, 63B, especially when the displays 63A, 63B are part of a display to be worn on the head. Moreover, there is the option of representing the recorded images as stereoscopic images on a large monitor that is observed by staff in the operating theater using suitable 3-D glasses. For the purpose of differentiating the partial stereoscopic images, the latter may be represented using e.g. different polarizations of the light emitted by the monitor during the display of the stereoscopic images on the monitor. The 3-D glasses then contain switchable polarizers that are switched synchronously with the display of the partial images on the monitor.

In particular, when the images are displayed on a monitor or in an HMD, surgical staff lose the direct relationship between the imaged object region and the position and orientation of the latter in the observation object 3—a relationship that they would have had if they were viewing the observation object with the aid of a binocular tube or a digital tube that is securely attached to the surgical microscope.

The illumination device 50 and its illumination beam path in the surgical microscope 2, 2′ are explained in more detail below with reference to FIGS. 4 and 5. FIG. 4 shows the main objective 5 of the surgical microscope 2, 2′ and the illumination device 50 with an illumination optics unit 45 that comprises a collector optics unit 51 and a condenser optics unit 53. In the present exemplary embodiment, both the collector optics unit 51 and the condenser optics unit 53 are constructed from lens groups in order to reduce imaging errors in the illumination beam path as far as possible. With the aid of the illumination optics unit 45, an illuminated field 57 is generated in an illuminated field plane 58. By means of a beam splitter, for example a partially transmissive mirror 43, the illumination beam path is input coupled into the main objective 5 of the surgical microscope 2, 2′ and directed via the main objective 5 at the observation object 3 such that the illuminated field plane 58 is located on the surface of the observation object 3 to be illuminated. In addition, FIG. 4 shows the optical elements 48 of the observation beam path and a camera 61 of the surgical microscope 2, 2′ in very schematic fashion.

In the present exemplary embodiment, the illumination device 50 of the surgical microscope 2, 2′ is designed as so-called Kohler illumination. In a Kohler illumination, the light source 41 is imaged by means of the collector optics unit 51 into an intermediate image plane that generally contains an aperture stop 55 which can be used to set, in a targeted manner, the brightness in the illuminated field 57 generated by the illumination device 50. Furthermore, a field stop 59 is present and arranged in a plane located between the collector optics unit 51 and the intermediate image plane with the aperture stop 55. The position of the plane of the field stop 59 is chosen such that it represents a plane conjugate to the illuminated field plane 58 in the illumination beam path. Structures that are located in one of two conjugate planes are imaged in focus in the other plane (cf. also FIG. 5). In the present exemplary embodiment, the illuminated opening of the field stop 59 is therefore sharply imaged into the illuminated field plane 58 by means of the condenser optics unit 53 in conjunction with the main objective 5. This imaging is shown schematically in FIG. 5, which shows the illuminated field plane 58, the field stop 59 located in the plane conjugate to the illuminated field plane 58 and, very schematically, the imaging optics unit formed by the condenser optics unit 53 and the main objective 5. The figure also shows the imaging beam paths for three selected field points 70A, 70B, 70C of the homogeneously illuminated aperture stop, via which beam paths the field points 70A, 70B, 70C located in the opening of the aperture stop 59 are imaged onto field points 71A, 71B, 71C in the illuminated field plane 58 by means of the condenser optics unit 53 and the main objective 5. Since the illuminated field plane 58 is generally located on the surface of the observation object 3, a sharply delimited illuminated field 57 with the image of the field stop 59 is formed on the surface of the observation object 3. The plane of the field stop 59 is also so far away from the plane of the aperture stop 55—and hence so far away from the image of the light source—that the field stop 59 can be illuminated homogeneously. The illuminated field 57 thus represents a homogeneously illuminated and sharply delimited illuminated field 57 on the observation object 3.

It should be noted that in the configuration shown in FIG. 4, the illumination beam path passes through the main objective 5, but this is not mandatory. Instead, as already mentioned, the illumination beam path may also be guided past the main objective 5 to the observation object 3. In this case, the condenser optics unit 53 is designed such that it can independently perform the imaging of the aperture stop into the illuminated field plane.

Since structures present in the plane of the field stop 59 are imaged into the illuminated field plane 58 in focus, it is possible to image a structure onto the illuminated field plane with the aid of a suitable stop. FIG. 6 shows a stop wheel 70 which in addition to a usual field stop 59 comprises structured stops 71, 73, 75 that can be introduced into the imaging beam path from FIG. 5 in alternation by rotating the filter wheel 70 about its rotation axis RA, as illustrated in FIG. 7. The structured stops 71, 73, 75 each have a structure that makes it possible on the basis of the projection of said structures onto the observation object 3 to indicate on the observation object 3 the orientation of the object region that was imaged on the image sensor 61. In the exemplary embodiment illustrated in FIG. 6, this is implemented by virtue of the orientations of a structured stop 71, 73, 75 introduced into the beam path corresponding to the orientation of the image sensor 61 in the surgical microscope 2, 2′ and by virtue of each structured stop 71, 73, 75 having four openings A, B, C, D that are separated from one another by bars 76. The intersection point 77 of the bars 76 in this case represents the center of the image sensor 61 and the outer edges 79 of the openings A, B, C, D represent the edge of the image sensor 61, with the outer edges 79 of the openings A, B, C, D being those edges that are not adjacent to the bars 76. The opening B also has a chamfered region 80 in the corner at which its two outer edges 79 would meet. This chamfered region 80 renders possible a clear identification of the orientation of the image sensor 61 in relation to the observation object 3 and hence a clear identification of the orientation of the object region in the observation object 3 imaged on the image sensor 61. When one of the structured stops 71, 73, 75 is introduced into the beam path, a structure in the form of a shadow image is projected onto the observation object 3 by the bars 76 and the chamfered region 80 within the illuminated field 57 and said structure indicates the orientation and the center of the image sensor 16 on the observation object 3 because the structured stop is in a plane conjugate to the illuminated field plane 58. Thus, together with the condenser optics unit 53 and the main objective 5 the structured stops 71, 73, 75 represent a projection device for projecting a structure onto the observation object 3.

If, as shown in FIG. 6, the structured stops 71, 73, 75 have different sizes, they can also provide an indication of the set magnification by way of the size of the structure projected on the observation object. It should be noted at this point that the chamfered region need not necessarily be present in opening B but may instead be present in any other opening. In addition, the structured stops need not have the structure shown in FIG. 6. Rather, they may have any structure that makes it possible to mark the center and the orientation of the object region recorded by the image sensor 61. Should the edge of the recorded object region moreover also be marked, such a structure must also have features by means of which the position of the edge of the object region recorded by the image sensor 61 can be identified. In addition, it is also not necessary for all structured stops on the stop wheel 70 to have the same structure. In this context, how the structure of a stop looks may be made dependent on for example the size of the shadow image projected onto the observation object 3 by means of the stop, for example in order to make small structures more recognizable.

In an alternative illumination device, by means of which a structure can be projected onto the observation object 3 and projected onto the observation object 3 with the aid of the illumination light that generates the illuminated field 57, a display 170 is arranged in the plane of the aperture stop in place of the stop wheel, and the pixels of said display can be switched either transparent or opaque and the structure to be projected can be displayed on said display. The display 170, which for example may be an LED display or an LCD display, is schematically shown in FIG. 8. The opaque pixels block the light from the light source, while the transparent pixels let it pass. Any structure can be displayed on the display 170 by appropriately controlling the pixels, and the display can serve as a structured stop with a freely configurable structure. In the exemplary embodiment shown in FIG. 8, the same structure as also used in the stop wheel 70 from FIG. 6 is displayed on the display 170. The transparent regions A, B, C and D of the display allow the light from the light source to pass. The remaining regions (depicted using hatching in FIG. 8) are opaque and block the light. As a result, the display 170 shows the same structure as was described with reference to FIG. 6, i.e. the opaque regions form bars 176 that meet at an intersection point 177 which represents the center of the image sensor 61 and hence the center of the object region imaged on the image sensor 61. The transparent regions form the openings A, B, C, D whose outer edges 179 represent the edge of the image sensor 61. The transparent region B has a chamfered region 180, on the basis of which the orientation of the image sensor 61 and hence the orientation of the object region imaged on the image sensor 61 is identifiable in relation to the observation object 3. Since the structure on the display 170 can be scaled freely, it can be adapted very precisely to the magnification used in the observation beam paths. In particular, accurate adaptation to a continuously variable magnification is also possible. It is also possible to use a color display. In that case, information may also be provided in a color-coded manner. For example, the chamfered region 180 may be omitted, and the opening B may instead be represented in tinted fashion. This also cancels the symmetry, and so the orientation of the image sensor 61 is clearly identifiable. A person skilled in the art identifies that there are a variety of ways to cancel symmetry in order to render an unambiguous orientation identifiable. In the context of the present invention, it is therefore only important that the projected structure has a broken symmetry.

As an alternative to the transilluminated display 170 in the plane of the aperture stop, there is the possibility of arranging a self-luminous display such as an OLED display in the plane conjugate to the illuminated field plane 58. This display then acts as a light source for the illumination beam path. Since said display is in a plane conjugate to the illuminated field plane 58, every image displayed on the display is projected into the illuminated field plane 58. A structure as may be realized by the structured stop or the transilluminated display may in that case also be realized using the self-luminous display, by virtue of displaying said structure on the self-luminous display. In this embodiment variant, the self-luminous display, the condenser optics unit 53 and the main objective 5 represent a projection device for projecting a structure onto the observation object 3.

A piece of optical observation equipment is often part of an optical observation system which, in addition to the piece of optical observation equipment, at least still comprises a positioning apparatus to which it is fastened. By means of the positioning apparatus, the piece of optical observation equipment can then be positioned and/or oriented in a suitable manner relative to the observation object to be observed. The suitable positioning and/or orientation may be brought about either manually or by motor. In the present exemplary embodiment, the surgical microscope 2, 2′ as a piece of optical observation equipment is fastened to a stand 201 with stand arms that are moveable relative to one another as a positioning apparatus. The stand 201 may be a motor-driven stand in particular, which allows positioning and/or orientation of the surgical microscope 2, 2′ by means of suitable actuators. Below, the stand 201 and the degrees of freedom made available by the stand for the surgical microscope 2, 2′ are described in more detail on the basis of FIGS. 9 and 10.

In the example of a stand 201 shown in FIG. 9, the stand rests on a stand base 205 which on its lower side has rollers 206 that allow a displacement of the stand 201. In order to prevent an unwanted displacement of the stand 201, the stand base 205 moreover comprises a foot brake 207.

As stand members, the actual stand 201 comprises a height-adjustable stand column 208, a support arm 209, a spring arm 210 and a microscope mount 211, which in turn comprises a connection element 213, a swivel arm 215 and a holding arm 214. The degrees of freedom provided by the stand members for positioning the surgical microscope 2, 2′ are shown in FIG. 10. At its one end, the support arm 209 is connected to the stand column 208 in a manner rotatable about an axis A. At the other end of the support arm 209, one end of the spring arm 210 is fastened in a manner rotatable about an axis B that is parallel to the axis A such that the support arm 209 and the spring arm 210 form an articulated arm. The other end of the spring arm 210 is formed by a tilt mechanism (not depicted here), on which the microscope mount 211 is fastened and which enables a tilting of the microscope mount 211 about the axis C.

The microscope mount 211 has an axis of rotation D, a swivel axis E and a tilt axis F, about which the microscope 2, 2′ can be rotated, swiveled and tilted, respectively. Using a connection element 213, the microscope mount 211 is fastened at the outer end of the spring arm 210 in a manner rotatable about the axis of rotation D. The axis of rotation D extends along the connection element 213. The connection element 213 is adjoined by a swivel arm 215, with the aid of which the surgical microscope 2, 2′, more precisely a holding arm 214 which is attached to the swivel arm 215 and on which holding arm the surgical microscope 2, 2′ is fastened by means of a microscope holder (not illustrated), can be swiveled about the swivel axis E. The swivel axis E extends through the swivel arm 215. The angle between the swivel arm 215 and the connection element 213, i.e. the angle between the swivel axis E and the axis of rotation D, can be varied by means of an adjustment mechanism arranged between the connection part 213 and the swivel arm 215.

The tilt axis F, which enables tilting of the surgical microscope 2, 2′ extends through the holding arm 214 in a manner perpendicular to the plane of the illustration. The surgical microscope 2, 2′ is fastened to the holding arm 214 by means of a microscope holder (not depicted here).

The degrees of freedom of the microscope mount 211 and the adjustment options of the surgical microscope 2, 2′, e.g. focusing, sharpness, magnification factor, etc., may be set by way of an adjustment device 202, which is illustrated as a foot control panel in the present exemplary embodiment. However, said adjustment device may also be realized as a hand-operated switching element or as a combination of both options mentioned. In addition to that or in an alternative, the adjustment device may moreover also include a keyboard and/or a touch display and/or a voice input unit.

In order to prevent an unwanted adjustment of the surgical microscope 2, 2′ from a selected position, the stand members or the joints between the stand members are provided with brakes 212 which are deactivated, i.e. released, for the purpose of positioning the surgical microscope 2, 2′ and are re-activated, i.e. fixed, after the surgical microscope 2, 2′ has been positioned.

The present exemplary embodiment provides for a controller 217 that receives from the stand 201 a status signal which indicates whether the brakes 212 are in a deactivated state or in an activated state. Should the status signal indicate that the brakes 212 are in a deactivated state, the controller 217 transmits an activation signal to the illumination device 50 of the surgical microscope 2, 2′, which activates the projection device on receipt of the activation signal. Deactivating the brakes 212 generally means that the surgical microscope 2, 2′ should be positioned and/or oriented or else repositioned and/or reoriented. During the positioning and/or orientation process, the projection of a structure as described previously helps the medical staff to identify on the observation object 3 the current position and/or orientation of the object region imaged on the image sensor 61. On the basis of the projected structure, the medical staff is then able to precisely position and/or orient the surgical microscope 2, 2′. The brakes 212 are reactivated again by the medical staff as soon as the positioning and/or orientation process has been completed. The status signal thereupon indicates an activated status of the brakes 212. As a result, the controller 217 transmits a deactivation signal to the illumination device of the surgical microscope 2, 2′, which deactivates the projection device again on receipt of the activation signal.

If the stand 201 is designed for the movement of the surgical microscope 2, 2′ by hand, a motion detector for detecting a movement of the surgical microscope 2, 2′ may be present in addition or in an alternative. For example, the motion detector may be an acceleration sensor. However, a tracking system that tracks the position of the surgical microscope 2, 2′ may also serve as a motion detector. In that case, the controller 217 is connected to the motion detector in order to receive a movement status signal representing the movement state of the surgical microscope 2, 2′ and to the illumination device in order to output an activation signal for activating the projection of the structure onto the observation object. In addition, it is configured in this case to output the activation signal only if the movement status signal represents a movement of the surgical microscope 2, 2′. To avoid an unintentional activation of the projection, e.g. due to vibrations, it is e.g. possible to store in the controller 217 a minimum duration over which the movement must be detected in order for the projection to be activated. In addition to that or in an alternative, one or more movement patterns that are characteristic, for example, for movements for positioning the surgical microscope 2, 2′ by hand may be stored in the controller 217. The automatic activation of the projection can then be limited to such detected movements that include one of the stored movement patterns.

The present invention has been described in detail on the basis of exemplary embodiments for explanatory purposes. On the basis of the description, a person skilled in the art recognizes that deviations from the exemplary embodiment are possible without departing from the scope of protection defined in the attached claims. For example, the number and configuration of the arms and joints of the stand may differ from those of the stand described with reference to FIGS. 9 and 10. All that is important is that the stand allows the position and/or orientation of the piece of optical observation equipment fastened to it to be changed. Therefore, the present invention is intended to be restricted only by the appended claims.

1. An optical observation system, comprising:
a piece of optical observation equipment having at least one imaging beam path that generates an image of an object region of an observation object, and an illumination device having:
at least one illumination beam path that leads from an illumination light source to an observation object and serves to generate an illuminated field on the observation object by illumination light, and
a projection device for projecting a structure onto the observation object, wherein the projection device acts on the illumination light in such a way that the structure is projected onto the observation object within the illuminated field with the aid of the illumination light that generates the illuminated field;
a positioning apparatus for positioning the piece of optical observation equipment in relation to the observation object, wherein the positioning apparatus comprises at least one brake which can be put into an activated and a deactivated state, wherein the brake in the activated state blocks a movement of the piece of optical observation equipment in relation to the observation object and does not block said movement in the deactivated state; and
a controller connected to the positioning apparatus in order to receive a status signal representing the state of the at least one brake and connected to the illumination device in order to output an activation signal for activating the projection of the structure onto the observation object, wherein the controller is configured to output the activation signal only if the status signal represents a deactivated state of the brake.

2. The optical observation system as claimed in claim 1, wherein the projection device is configured to act on the illumination light in such a way that there is a structure-creating shadowing in the illuminated field.

3. The optical observation system as claimed in claim 2, wherein the shadowing is brought about by a stop that is situated in the illumination beam path and arranged in or near to a plane of the illumination beam path conjugate to the illuminated field plane of the illuminated field.

4. The optical observation system as claimed in claim 3, wherein a stop changer device having a plurality of stops that differ from one another in terms of their shape and/or their size is present in or near to the plane of the illumination beam path conjugate to the illuminated field plane of the illuminated field.

5. The optical observation system as claimed in claim 1, wherein a self-luminous display is present as the at least one illumination light source and arranged in or near to a plane of the illumination beam path conjugate to the illuminated field plane of the illuminated field, and the structure is displayed on the display.

6. (canceled)

7. (canceled)

8. The optical observation system as claimed in claim 1, wherein the size of the structure projected onto the observation object represents an indicator for the object region that is imaged on the image sensor.

9. The optical observation system as claimed in claim 1, wherein the structure projected onto the observation object contains a marking, by which it is possible to identify the orientation of the image sensor in relation to the observation object.

10. (canceled)

11. (canceled)

12. A method for identifying on an observation object an object region of said observation object that has been imaged on an image sensor of a piece of optical observation equipment, wherein a structure is projected onto the observation object with the aid of illumination light that generates an illuminated field on the observation object, wherein the structure represents an indicator for the object region of the observation object imaged on the image sensor, and the structure is only projected onto the observation object when the piece of optical observation equipment (2, 2′) is moved.

13. The method as claimed in claim 12, wherein the structure is projected onto the observation object with the aid of shadowing in the illuminated field.

14. The method as claimed in claim 12, wherein the shadowing in the illuminated field is brought about by a stop in or near to a plane of the illumination beam path conjugate to the illuminated field plane of the illuminated field.

15. The method as claimed in claim 12, wherein the structure is only projected onto the observation object when at least one brake of a positioning apparatus for positioning and/or orienting the piece of optical observation equipment in relation to the observation object is deactivated.

16. The method as claimed in claim 12, wherein the projecting of the structure onto the observation object is terminated when all brakes are activated.

17. (canceled)

18. An optical observation system, comprising:
a piece of optical observation equipment having at least one imaging beam path that generates an image of an object region of an observation object, and an illumination device having:
at least one illumination beam path that leads from an illumination light source to an observation object and serves to generate an illuminated field on the observation object by illumination light, and
a projection device for projecting a structure onto the observation object, wherein the projection device acts on the illumination light in such a way that the structure is projected onto the observation object within the illuminated field with the aid of the illumination light that generates the illuminated field,
a positioning apparatus for manually moving the piece of optical observation equipment in relation to the observation object,
a motion detector for detecting a movement of the piece of optical observation equipment, and
a controller connected to the motion detector in order to receive a movement status signal representing the movement state of the piece of optical observation equipment and connected to the illumination device in order to output an activation signal for activating the projection of the structure onto the observation object, wherein the controller is configured to output the activation signal only if the movement status signal represents a movement of the piece of optical observation equipment.

19. The optical observation system as claimed in claim 18, wherein the projection device is configured to act on the illumination light in such a way that there is a structure-creating shadowing in the illuminated field.

20. The optical observation system as claimed in claim 19, wherein the shadowing is brought about by a stop that is situated in the illumination beam path and arranged in or near to a plane of the illumination beam path conjugate to the illuminated field plane of the illuminated field.

21. The optical observation system as claimed in claim 20, wherein a stop changer device having a plurality of stops that differ from one another in terms of their shape and/or their size is present in or near to the plane of the illumination beam path conjugate to the illuminated field plane of the illuminated field.

22. The optical observation system as claimed in claim 18, wherein a self-luminous display is present as the at least one illumination light source and arranged in or near to a plane of the illumination beam path conjugate to the illuminated field plane of the illuminated field, and the structure is displayed on the display.

23. The optical observation system as claimed in claim 18, wherein the size of the structure projected onto the observation object represents an indicator for the object region that is imaged on the image sensor.

24. The optical observation system as claimed in claim 18, wherein the structure projected onto the observation object contains a marking, by which it is possible to identify the orientation of the image sensor in relation to the observation object.
